(19) United States
(12) Patent Application Publication
(10) Pub. No.: US 2023/0218302 A1
(43) Pub. Date: Jul. 13, 2023

(54) DETACHMENT MECHANISM FOR DETACHABLE-TIP ENDOVASCULAR DEVICES

(71) Applicant: Icahn School of Medicine at Mount Sinai, New York, NY (US)

(72) Inventors: Daniel VILLARROEL, New York, NY (US); Kurt YAEGER, New York, NY (US); Wasem KHALID, New York, NY (US); Joseph Allan BORRELLO, New York, NY (US)

(21) Appl. No.: 18/009,017

(22) PCT Filed: Jun. 15, 2021
(86) PCT No.: PCT/US2021/037323

§ 371 Date: Dec. 8, 2022

Related U.S. Application Data
(60) Provisional application No. 63/038,955, filed on Jun. 15, 2020.

(51) Int. Cl. A61B 17/12

(52) U.S. Cl. CPC A61B 17/12136; A61B 17/12113; A61B 2017/00867; A61B 2017/12077

(57) ABSTRACT

An endovascular device includes a catheter and a detachment mechanism that comprises a shape memory material located at a distal end of the catheter. The device further includes a detachable tip component secured by the detachment mechanism, wherein the detachment mechanism is configured such that application of electricity to the detachment mechanism causes the detachment mechanism to open and release the detachable tip component.

### CROSS REFERENCE TO RELATED APPLICATION

The present application is based on and claims priority to U.S. Provisional Pat. Application 63/038,955, filed Jun. 15, 2020, and is related to U.S. Pat. Application No. 62/799,409, filed Jan. 31, 2019, and International patent application No. PCT/US2020/016208, filed Jan. 31, 2020, the entire contents of each application is incorporated by reference herein as if expressly set forth in its respective entirety herein.

### TECHNICAL FIELD

The present invention relates to surgical devices and methods of treatment and more particularly, relates to detachable-tip endovascular devices that each has a detachable tip component that is held in place by a detachment mechanism that is controllable to release the detachable tip component. One exemplary detachable-tip endovascular device comprises a detachable tip balloon microcatheter for embolization of vascular territories; however, the devices can equally be configured to treat other conditions.

### BACKGROUND

Detachable endovascular devices have been used to treat aneurysms, vascular tumors, vessel injuries, arteriovenous malformations (AVM), and dural arteriovenous fistulas (dAVF) for decades. At present, there is a variety of endovascular detachment and deployment mechanisms, which include but are not limited to general categories of mechanical, chemical, and electrolytic. Throughout the years detachable endovascular devices have become highly specialized and increasingly effective as a means of treating vascular pathology; however, these devices have limitations associated with existing detachment mechanisms, and can be improved upon.

Purely mechanical detachment systems are the oldest form of endovascular separation and are the most widely used. Breakaway joints in the detachment zone are designed in various forms, some of which are heat shrink sheaths joining a detachable tip to the catheter body, screw-like mating of detachable tip directly to the catheter body, as well as press fitted tip directly into the catheter body. Although they differ in appearance, each of these systems requires force to be transmitted across the catheter body through the joint to facilitate breakage. Early devices used hydrostatic pressure to detach the tip from the body of the device whereas many modern devices use tensile force. Mechanical detachment systems in endovascular devices have breakage thresholds between 10 to 160 grams depending on the sensitivity of the device and the degree of embodiment in the embolic agent. In the Apollo microcatheter, commonly used to treat brain arteriovenous malformations (AVMs) via injection of liquid embolic agents, a tensile force of 33 grams is necessary to detach the embedded tip from the catheter body.

Although mechanical attachment systems are widely used and have proven to be reliable, their successful detachment is dependent on a tensile force running through the joint to the embedded tip. The issues that this may result in are twofold. First, pulling on a catheter that is adhered to a solidified embolic agent cast within distal vasculature can cause shifting of the proximal vessels, which can lead to avulsion of small branches, leading to tissue hemorrhage or ischemia. The secondary issue in mechanical endovascular detachment system is that as force is applied along the catheter the catheter body attempts to straighten out relative to the points of attachment (the surgeon and the embedded tip). This puts excess strain/ shear on the vasculature and may potentially harm healthy tissue. To avoid excess trauma and complications during surgery there needs to be a detachment mechanism that requires minimal force. However, setting this minimum force too low can lead to premature tip detachment, leading to foreign body deposition in the vasculature, which in itself cause tissue ischemia.

Chemical detachment mechanisms are less common. These systems use a dissolvable adhesive to fix the detachable tip to the catheter body. In this system very little force is needed to detach an embedded tip because a solvent will break the bond allowing the tip to effortlessly slide off the catheter body. An example of this type of system is the Sonic microcatheter, also used to inject liquid embolic agents to treat AVM. The Sonic microcatheter is not approved by the FDA and is not available in the USA. The joint in this system is made of a DMSO soluble adhesive. When injecting the embolic agent / DMSO mixture the bond will dissolve and break apart effortlessly allowing for a clean extraction of the catheter body.

Since chemical-based detachments systems do not need to be pulled or pushed apart there is no transmission of force form the surgeon to the malformation, the potential risk of damage to the vasculature due to excess strain/ shear is avoided. Although chemical-based detachment systems successfully decrease the amount of force needed to break away the tip from the catheter body they are considerably less reliable than mechanical detachment systems. Chemical detachment systems similar to what is used in the Sonic device have a risk of premature detachment, which as mentioned, can result in complications.

Electrolytic endovascular detachment mechanisms are typically seen in embolic coils or intra-saccular devices both used for aneurysm embolization. Embolic coils are soft flexible coils of platinum that are fused to a stainless-steel joint that fixes the coil to a stiffer pusher wire. The coils are packing into an aneurysm dome to occlude it. Once a coil is implanted into a aneurysm dome, a 2 mA current oxidizes the stainless steel joint , which breaks down releasing the platinum coil from the pusher wire. Detachment via Electrolysis requires no force to be transferred across the catheter body nor does it require interactions between and adhesive and solvent. This means that not only is it an effective mode of detachment it is also stable and reliable. Although Electrolytic detachment systems have all the aspects of being an ideal endovascular detachment mechanism, its use is far too specified as it is currently primarily used in detaching embolization coils or intra-saccular devices into aneurysm domes.

Each of the conventional modes of detachment are currently used and operate with efficiency. Yet there are still limitations that make them insufficient as endovascular detachable devices. What is needed is an effective, reliable, and versatile detachment mechanism, with on demand detachment, to be used across a broad spectrum of endovascular devices.

### SUMMARY

The present disclosure sets forth a general endovascular electrical detachment mechanism for use in separating distal elements (e.g., a detachable tip component) from a proximal delivery system (e.g., a catheter). In one case, described herein, the distal element is a detachable tip that can detach on demand when a current is applied to the system. This is often needed during endovascular embolization of vascular abnormalities (aneurysms, arteriovenous malformations, arteriovenous fistulae) with injection of liquid embolic agents, that occasionally harden onto the catheter, preventing removal from the body. Other embodiments of this electric detachment may take the form of separating distal embolic devices from a proximal catheter, such as detachable balloons or intrasaccular devices for aneurysm embolization or sacrifice of a vessel. We describe a mechanism in which a nitinol wire is wrapped around the distal detachable device, tightly wound to secure said device, but which loosens with thermal energy as caused by a current sent from the proximal catheter. This thermal decoupling allows loosening of the nitinol wire around the distal device, permitting easy removal. This atraumatic, on-demand mechanism is safer and more reliable than currently available endovascular detachment mechanisms, and has a wide range of possible embodiments.

In one exemplary embodiment, an endovascular device includes a catheter and a detachment mechanism comprising a shape memory material located at a distal end of the catheter. The endovascular device includes a detachable tip component that is detachably secured to the catheter by the detachment mechanism. The detachment mechanism is configured such that application of electricity to the detachment mechanism causes the detachment mechanism to open and release the detachable tip component.

In one embodiment, the detachable tip component comprises an embolic device and the catheter comprises a detachable tip balloon microcatheter that includes a detachment mechanism that comprises a shape memory material located at a distal end of the catheter. The embolic device is secured by the detachment mechanism which is configured such that application of electricity to the detachment mechanism causes the detachment mechanism to open and release the embolic device. The shape memory material can be nitinol. The detachment mechanism can comprise a wire and have a coil (coiled portion) that is formed of the shape memory material. The coil surrounds the embolic device and is distally spaced from the catheter. In a rest position of the detachment mechanism, when the shape memory material, including the coil, is not under influence of electricity, the coil tightly grasps the embolic device, which passes through an opening of the coil, and maintains coupling between the detachable tip component, such as an embolic device, and the catheter and wherein in an actuated state when electricity is applied, the coil expands and opens, thereby releasing the detachable tip component and severing the coupling between the detachable tip component and the catheter.

### BRIEF DESCRIPTION OF THE DRAWING FIGURES

FIG. 1 is a side elevation view of a detachable tip sheathed balloon microcatheter according to one exemplary embodiment;

FIG. 2 is cross-sectional view taken along the line A-A of FIG. 1;

FIG. 3 is a side perspective view of a detachable-tip balloon microcatheter system including the detachable tip sheathed balloon microcatheter of FIG. 1 and the proximal hub detachment mechanism;

FIG. 4 is another side view of the detachable-tip balloon microcatheter system;

FIG. 5 is a side view of the detachable tip sheathed balloon microcatheter in a delated state;

FIG. 6 is an exploded view of the deflated balloon microcatheter with the detachable tip assembly being shown detached from the main catheter portion;

FIG. 7 is a cross-sectional view of the detachable tip sheathed balloon microcatheter;

FIG. 8 is a side perspective view of the detachable tip sheathed balloon microcatheter according to another embodiment with an extended protective balloon sheath;

FIG. 9 is a side perspective view of the detachable tip sheathed balloon microcatheter according to another embodiment with a protective balloon sheath that covers the entire balloon;

FIG. 10 is a side perspective view of the detachable tip sheathed balloon microcatheter according to another embodiment with a protective balloon sheath that includes impinged strips;

FIGS. 11A-11H shows exemplary steps for transarterial or transvenous embolization of AVMs using the proposed novel device mechanism;

FIGS. 12A-12D are cross-sectional views of alternative detachable tip sheathed balloon microcatheters;

FIGS. 13A-13D are perspective views of the alternative detachable tip sheathed balloon microcatheters of FIGS. 12A-12D;

FIG. 14 is a side elevation view of a detachable tip balloon microcatheter with a detachment mechanism according to one exemplary embodiment;

FIG. 15 is an enlarged side elevation view of a portion of microcatheter illustrating the detachment mechanism;

FIG. 16 is an angled rear perspective view of the microcatheter;

FIG. 17 is a rear perspective view of a section of the microcatheter showing a wire that that is part of the detachment mechanism;

FIG. 18 is a perspective view of an exemplary rotating hemostasis valve (RHV) in which a flush line can be configured to have an actuator to activate the wire of the detachment mechanism;

FIG. 19A is a side perspective view of a detachable tip sheathed balloon microcatheter according to one exemplary embodiment;

FIG. 19B is a side perspective view of a portion of the microcatheter of FIG. 19A;

FIG. 20A is a side elevation view of the microcatheter of FIG. 19A;

FIG. 20B is a side cross-sectional view of the microcatheter;

FIG. 21 is a side perspective view showing the detachable tip in an attached state with the microcatheter and the detachment mechanism is in a first state (closed condition); and

FIG. 22 is a side exploded perspective view showing the detachable tip detached from the microcatheter and the detachment mechanism is in a second state (open condition).

### DETAILED DESCRIPTION OF CERTAIN EMBODIMENTS

As previously mentioned, detachable endovascular devices have been used to treat aneurysms, vascular tumors, vessel injuries, arteriovenous malformations (AVM), and dural arteriovenous fistulas (dAVF) for decades. At present, there is a variety of endovascular detachment and deployment mechanisms, which include but are not limited to general categories of mechanical, chemical, and electrolytic. Throughout the years detachable endovascular devices have become highly specialized and increasingly effective as a means of treating vascular pathology; however, these devices have limitations associated with existing detachment mechanisms, and can be improved upon.

### Arteriovenous Malformations (AVM)

Given the relatively low procedural morbidity with endovascular techniques (compared to surgical resection), AVM embolization is typically first-line therapy and is the process of delivering an artificial embolic agent to completely occlude the AVM and can be followed by microsurgical resection or surgery (e.g., radiosurgery) if deemed amenable.

Currently, liquid embolization is technically conducted using two different types of embolic agents, some adhesive and some non-adhesive; and different classes of catheters: non-detachable catheters, detachable-tip microcatheters, and balloon microcatheters.

### Embolic Agents

Embolic agents frequently utilized are liquids, particulates, coils, occluding plugs, and balloons. Some embolic agents have a short lived action such as collagen and gelfoam, whereas others, like glue or coils, are permanent. Liquid embolic agents include, Onyx ™, alcohol, ALGEL, and Phil ™. N-Butyl Cyanoacrylate (NBCA, glue) has also been utilized as a permanent embolic agent. NBCA is diluted with Ethiodol and tantalum. NBCA displays a fast polymerization rate when exposed to the ionic environment of blood. Ethiodol is used as vehicle and a polymerization retardant. Onyx ™, a mixture of ethylene alcohol vinyl polymer (EVOH), dimethyl sulfoxide (DMSO) and tantalum powder for radiopaque visualization, has been approved by the FDA for embolization of cerebral AVMs.

The solvent for Onyx ™ embolization is dimethyl sulfoxide (DMSO). DMSO prevents Onyx™ premature hardening in the catheter. During the embolization procedure the clinician injects DMSO when the catheter is in position. Consequently, Onyx™ is injected, moving the column of DMSO towards the distal catheter tip. When Onyx™ comes in contact with blood, DMSO diffuses away and the hardening process begins. In contrast with NBCA, Onyx ™ requires DMSO compatible catheters. NBCA is an adhesive agent and Onyx ™ is cohesive and non-adhesive, acting like lava and displaying progressive solidification and cohesiveness hardening from the inside out. Importantly, due to its cohesive nature, Onyx™ allows for slower injection times. However, both Onyx™ and glue can induce entrapment of the catheter in the vessel if not used properly.

Due to the tortuous nature of AVMs, the rate of complete obliteration of the lesion is approximately 20% and complications may result as architecturally complex AVMs require a greater level skill by the interventionalist. AVMs are dynamic structures with multiple arterial feeders. Embolization of one feeder inevitably affects blood flow in adjacent feeders as well as the pressure at the nidus; abrupt changes in pressure at the nidus increase hemorrhage risk. The angioarchitecture of the AVM determines the concentration of Onyx™ and embolization success requires selection of the Onyx ™ product with the appropriate viscosity. After selection of the Onyx™ product and DMSO catheter pre-treatment, the clinician must identify the best position of the catheter tip with respect to the AVM nidus to optimize treatment; inadequate penetration of the nidus will lead to ineffective embolization, in turn, over-penetration may increase nidus pressure leading to hemorrhage. The clinician must deliver the embolic agent at the precise rate and speed to achieve maximal penetration of the nidus while preventing excessive reflux. Generally, an accepted level of reflux is 1 to 2 cm. When deciding the embolizing agent delivery rate, the clinician must balance the need to provide a comprehensive embolization of the AVM while considering adjacent vasculature or distal compromise to the venous drainage of the nidus. Onyx™ injection occurs in two phases: the injection phase and the rest phase, each lasting between 30 and 120 seconds. Faster injection times can cause reflux, angionecrosis, and vasospasm, whereas longer resting phases might cause unintentional occlusion of the catheter. A clinician must decide when to stop the embolization and remove the catheter to avoid entrapment or fracturing the cast created by the embolic agent. The risk of catheter entrapment is 4% for Onyx™ embolization. With current techniques, precise timing of actions during embolization is critical to limit complications. Given these factors, achieving a complete endovascular cute rate is rare. Thus, while endovascular embolization is safer and less morbid than surgery, surgical resection has traditionally been considered curative, and endovascular treatment adjuvant.

The following items have been identified as areas needing improvement during embolization: 1) reducing embolic agent reflux (which is associated with stroke risk); 2) reducing force for detachment following embolization; 3) increasing speed of embolization procedures resulting from continuous embolization, (thereby improving nidus penetration and reducing the chance of complication); 4) reduced risk of catheter entrapment; and, 5) reduced procedure cost (due to reduced need for other single-purposed catheters); 6) improved health outcomes (given the shorter procedural time and less time under anesthesia); 7) reduced radiation exposure.

The most commonly utilized microcatheters in the market utilize glue and/or Onyx™ and are either in the form of detachable-tip microcatheters or non-detachable tip balloon microcatheters.

### Detachable-Tip Microcatheters

The detachable-tip microcatheter was developed as a solution to potential catheter entrapment during use of non-adhesive DMSO-compatible liquid embolic agents such as Onyx™, Squid or PHIL, and can be used for better embolization with adhesive liquid embolic agents such as IBCA or NBCA. The main feature of detachable tip microcatheter is that the distal section of the catheter incorporates a detachment zone that allows separation of the catheter when the catheter main body is retracted. There are currently two available detachable tip microcatheter systems for embolization in the market: the SONIC ™ (BALT, Montmorency, France, not approved by the FDA and not available in the USA) and the Apollo ™ (Medtronic).

Both the Apollo ™ and SONIC ™ microcatheters utilize radiopaque marker bands to visualize position in the vasculature and the length of detachment zone. The clinician utilizes the markers to estimate the extent of reflux relative to the embolic length of the detachment zone. The available detachable tip lengths are available between 15 mm and 50 mm. Both microcatheters are DMSO compatible. In the SONIC microcatheter, the connecting portion of catheter and detachable tip is DMSO soluble and therefore dissolves after injection of DMSO and embolic agent. In the Apollo microcatheter, detachment is achieved by gentle retraction of the main catheter body. The Apollo system tip detaches with a minimal, atraumatic force of 33 grams. Detachable-tip microcatheters are advantageous in situations where successful embolization would result in a significant cast around the tip of the catheter. As previously described, successful utilization of the detachable-tip microcatheters requires the formation of a plug proximal to the microcatheter tip before the Onyx™ or other embolic agent can move forward towards the AVM nidus. This allows the proceduralist to inject embolic material with enough velocity to occlude distal segments. In microcatheters without a detachable tip, reflux control depends on operator expertise to prevent catheter entrapment in the Onyx™ cast. However, the introduction of microcatheters with a detachable tip allows proceduralists to incorporate the catheter injection lumen into the Onyx™ proximal plug, permitting faster injection velocities of the embolic agent.

### Balloon Catheters

Balloon microcatheters optimize flow control in liquid embolization procedures. Compared to detachable-tip microcatheters, balloon assisted embolization allows more precise delivery of embolization agents preventing reflux and spillage of the embolic agent into non-target vessels. The Scepter C ™ and the Scepter XC ™ (Microvention, Inc, Tustin CA USA) are DMSO compatible, and thus can be used for embolization with Onyx™. However, they are incompatible for use with NBCA, given the glue’s adhesive interaction with catheters. The Scepter C ™ and the Scepter XC ™ are two of the more commonly used balloon microcatheters for liquid embolization procedures. However, there are other balloon microcatheters, such as the Balt Extrusions Eclipse catheter, that are not FDA approved for use in the US. Balloon microcatheters are coaxial, double-lumen access microcatheters that are DMSO-compatible and hydrophilic-coated. The double lumen allows for concurrent inflation of the balloon and delivery of liquid embolic agents or coils. The tip length is shorter, measuring approximately 5 mm. Radiopaque markers located at the distal catheter tip and the distal balloon end indicate the length of the catheter tip.

The balloon portion of the Scepter C ™ acts as a plug to ensure flow control of the embolic agent from another, adjacent microcatheter into an AVM nidus. The proximal plug created by balloon inflation decreases the procedure time required by detachable-tip microcatheters, as it obviates the need to create an obstructive Onyx ™ cast. However, it has been observed that unwanted retrograde flow or reflux of Onyx™ can still occur requiring the clinician to increase the size of the balloon or to temporarily stop the injection procedure. Furthermore, as previously described, retrograde flow of Onyx ™ can potentially lead to catheter entrapment.

### Dual Microcatheter Techniques

Today, many embolization procedures are conducted with a dual microcatheter technique, leveraging the advantages of both the detachable-tip and balloon microcatheters. This combination technique is used for both transarterial and transvenous AVM embolization. In this technique, the two microcatheters are advanced alongside each other. The balloon microcatheter is inflated to provide distal flow control and proximal reflux prevention. The detachable-tip microcatheter is advanced more distally into the nidus. Subsequently, a plug is created by the distal catheter, either with metal coils or Onyx™. Nidus embolization is then achieved using the more distal detachable-tip microcatheter. When the Onyx™ cast hardens, the tip is detached, the balloon is deflated, and both catheters are removed. This technique requires more operator experience and surgical time compared to a single microcatheter technique but leads to a safer procedure and a more completely embolized AVM.

The advantage of balloon microcatheters versus non-balloon microcatheters, is the ability of balloons to arrest flow, and prevent reflux of a liquid embolic agent. The advantage of a detachable tip is its ability to be used with adhesive agents that permanently bind with the catheter but can be detached from the catheter body following injection.

In non-detachable tip microcatheters, after reaching the target, an occlusive embolic plug must be initially formed at the inflow vessel for proximal flow control and prevention of embolic material reflux into normal vasculature. Comparatively, inflation of a proximal balloon microcatheter allows improved flow control, such that injection of embolic agent can better perfuse the malformation. However, these catheters can become embedded in the embolic agent cast, requiring significant mechanical force for removal. This tension force can dislodge the cast or avulse small perforating blood vessels leading to ischemic end-organ complications.

### Detachable-Tip Sheathed Balloon Microcatheter System

As will be readily understood in view of the preceding discussion concerning the deficiencies of the traditional commercially available devices for performing embolization of AVMs or other vascular pathologies, devices disclosed herein overcome those deficiencies, and more particularly, the figures illustrate a single-body, detachable-tip balloon microcatheter system (assembly) 100 that can be used to treat any number of conditions in which detachment of the tip is required. For example, in one non-limiting example, the system 100 can be used for transarterial and transvenous embolization of AVMs or other vascular pathologies. In such application, the detachable-tip balloon microcatheter system 100 is configured to arrest flow and prevent reflux and reduce surgery time by promoting a more continuous and comprehensive embolization while reducing radiation exposure, risks and procedural complications. In addition, and as described in more detail below, the detachable, balloon-protective sheath permits the use of balloons with tissue adhesive embolic agents, such as NBCA, which is not possible with current, unprotected balloon microcatheters. The technology described in the present application represents a significant improvement over the currently available solutions by incorporating a balloon, a detachable tip segment, and a balloon-protective sheath. As described herein, the detachable-tip balloon microcatheter system 100 achieves these objectives.

As described in detail herein, the detachable-tip balloon microcatheter system 100 has the following advantageous technical features: 1) a detachable tip with covering sheath that protects the balloon from interaction with the embolic agent and facilitates the formation of an embolic cast. The sheath can be physically constructed as a super-elastic thin-walled tube that surrounds a portion of the balloon while being fixed to the detachable tip catheter; and 2) distal tip detachment mechanism: the detachable tip is press-fit into the balloon microcatheter lumen to make contact with a separate inner catheter. A novel sliding retract-release mechanism in the proximal hub operated by the surgeon retracts the balloon catheter while keeping the inner-catheter and detachable tip fixed, causing the atraumatic release of the tip (with minimal strain on the vessels).

As previously mentioned, the present application is related to previous patent application filings and therefore, additional details concerning exemplary detachable-tip balloon microcatheter systems 100 are disclosed in U.S. Pat. application No. 62/799,409 and International patent application No. PCT/US2020/016208. It will be readily understood that the detachable-tip balloon microcatheter system 100 is merely one exemplary type of device that can be used with the detachment mechanism that is described herein.

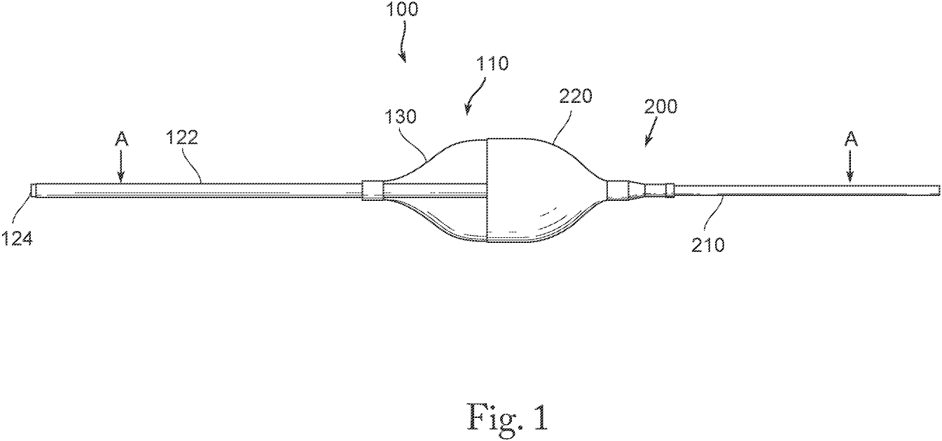

FIGS. 1-9 illustrate the detachable balloon microcatheter system 100 that includes several components (parts) that mate together to form the assembled system 100. In particular and according to one non-limiting example, the detachable balloon microcatheter system 100 includes a detachable tip sheathed balloon microcatheter 110 that is formed of a first part 120 in the form of a detachable balloon microcatheter that also can be considered to be a proximal part of the detachable tip sheathed balloon microcatheter 110 and a second part that is discussed in more detail and can be considered to be a distal part of the detachable tip sheathed balloon microcatheter 110. The first and second parts of the detachable tip sheathed balloon microcatheter 110 are shown separated from one another in the exploded view of FIG. 6.

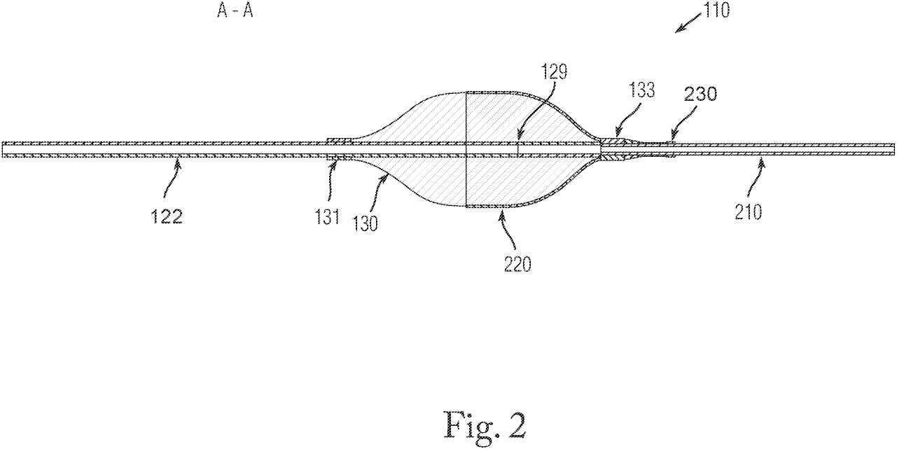

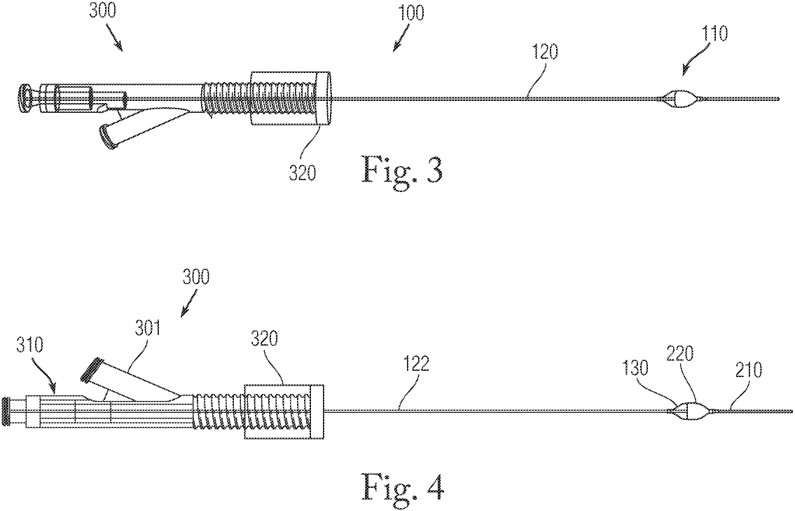

FIG. 1 is a side elevation view of the detachable tip sheathed balloon microcatheter 110, while FIG. 2 is a cross-sectional view of the detachable tip sheathed balloon microcatheter 110. FIGS. 3 and 4 illustrate the entire detachable balloon microcatheter system (assembly) 100 including the detachable tip sheathed balloon microcatheter 110.

The detachable balloon microcatheter 120 (first part of the detachable tip sheathed balloon microcatheter 110) comprises a dual catheter tube assembly (a catheter shaft) that generally has a proximal end and an opposing distal end. The dual catheter tube nature of the detachable balloon microcatheter 120 results from the fact that it includes an outer catheter tube (outer catheter) 122 and a separate inner catheter tube (inner catheter) 124 that is inserted into a central lumen of the outer catheter tube 122. The outer catheter tube 122 and inner catheter tube 124 can thus be concentric with one another.

The detachable balloon microcatheter 120 has an inflatable member, such as an inflatable balloon 130 that is coupled to the outer catheter tube 122 and surrounds a portion of the outer catheter tube 122 and more particularly, the inflatable balloon 130 extends along a length of the distal portion of the outer catheter tube 122. As illustrated, the inflatable balloon 130 is attached at both its ends (proximal and distal ends) to the outer catheter tube 122.

Any number of different balloon configurations and materials (including compliant materials) can be used for the balloon 130. As described herein, the inflatable balloon 130 is inflated and deflated as a result of delivering of a fluid (most often a liquid) to the interior of the inflatable balloon 130 and conversely, the inflatable balloon 130 is deflated as a result of removal of the fluid from the interior of the balloon as discussed below.

For example, the balloon 130 can be made of any standard balloon materials such as PET, nylon, polyurethane, silicone, PEBAX, etc. although a low-compliance material is preferred to prevent risk of over-expansion of the balloon. The balloon 130 optimally is made of a material with optimal hysteresis and optical (maximal) elasticity to decrease the profile and return to its minimal deflated state. Also, in preferred embodiments, the balloon 130 is formed of a material that is DMSO and acrylic glue compatible.

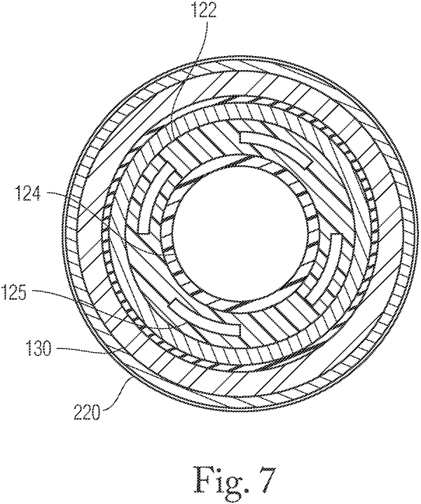

The outer catheter tube 122 has a multi lumen construction in that it may contain multiple lumens or a co-axial double catheter design. In one embodiment, the outer catheter tube 122 includes a central lumen in which the inner catheter tube 124 is inserted and there are one or more and preferably several surrounding channels 125 (FIG. 7) for balloon inflation (fluid flowing within channels 125 flow into/out of the interior of the balloon 130). As will be readily understood, the lumens and channels in the outer catheter tube 122 can vary in their constructions and more particularly, the sizes (e.g., diameters) and/or shapes of the lumens/channels can vary. For example, one lumen/channel can have a greater diameter due to fluid carrying considerations, etc. Embolic agent is delivered through the inner catheter tube 124 (by flowing through the lumen thereof). In other words, the embolic agent is delivered centrally through the detachable balloon microcatheter 120.

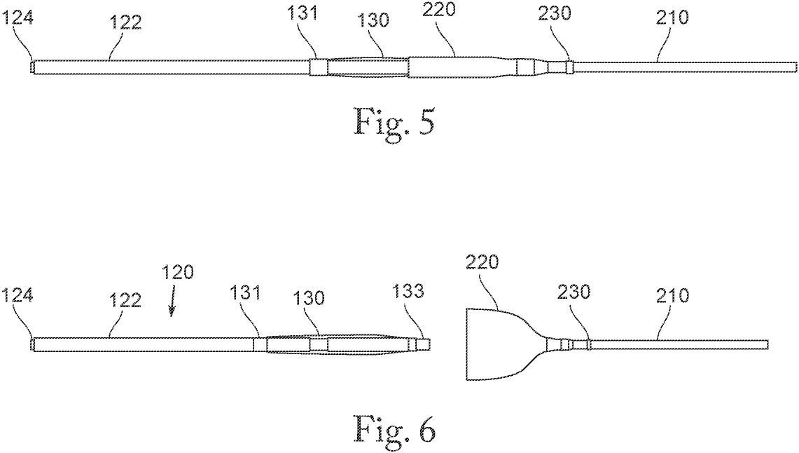

As shown in FIG. 7, the balloon 130 is concentrically arranged relative to the outer catheter tube 122 and the inner catheter tube 124. The balloon 130 thus, at least in part, defines the distal end of the detachable balloon microcatheter 120. As shown in FIGS. 5 and 6, the inflatable balloon 130 can be placed in a fully deflated state (FIGS. 5 and 6) and a fully inflated state (FIGS. 1 and 2).

The detachable balloon microcatheter 120 includes a detachable tip assembly 200 (e,g., an embolic device) that is formed at the distal end and represents the second part of the detachable tip sheathed balloon microcatheter 110. As described herein, the detachable tip assembly 200 is detachably coupled at its proximal end to the distal end of the detachable balloon microcatheter 120.

The detachable tip assembly 200 comprises a microcatheter tube 210 and a balloon sheath 220 and, as previously mentioned, the detachable tip assembly 200 is configured to be fitted into the distal end of the outer catheter tube 122 that is part of the detachable balloon microcatheter 120. More specifically, the proximal end portion of the microcatheter tube 210 is inserted into the distal end of the outer catheter tube 122 so as to form a press-fit (mechanical coupling) between the two (i.e., the outer diameter of the microcatheter tube 210 is only slightly less than the inner diameter of the outer catheter tube 122 resulting in the friction fit (press fit)). The inner catheter tube 124 does not extend completely to the distal end of the outer catheter tube 122 but is offset therefrom. This offset allows for the microcatheter tube 210 to be inserted.

The detachable tip assembly 200 can make contact with inner catheter tube 124 at a contact point, generally indicated at 129 somewhere near mid-length of balloon 130 and as a result, and more particularly, the proximal end of the microcatheter tube 210 abuts the distal end of the inner catheter tube 124 at contact point 129 so as to define a central continuous flow path for carrying and delivering the embolic agent. Thus, a coaxial, seamless connection is formed between the tubes 210, 124. It will be understood that the microcatheter tube 210 is approximated but not mechanically attached in view of the fact that the inner catheter tube 124 and the microcatheter tube 210 separate when the detachment mechanism is deployed. The inner catheter tube 124 and the microcatheter tube 210 can have the same dimensions (diameters) and can be reversibly connected in using a suitable technique. Thus, when the detachable tip assembly 200 engages the detachable balloon microcatheter 120, embolic agent is delivered through the inner catheter tube 124 to the microcatheter tube 210.

The balloon 130 can be constructed from a tube sealed to the outer catheter tube 122 through radiopaque ring seals 131 and 133 and connected via holes to balloon lumens/channels 125 for inflation.

The microcatheter tube 210 is thus open at both of its ends to allow the embolic agent to flow from the inner catheter tube 124 into and along the microcatheter tube 210 before being discharged from the microcatheter tube 210 at its open distal end.

The balloon sheath 220 can be an ultra-elastic tube permanently attached to the outer surface of the microcatheter tube 210 via a radiopaque seal 230 and extends proximally to cover at least half of the length of the balloon 130. The opposite end of the balloon sheath 220 is an open end that at least partially surrounds the microcatheter tube 210 (with a portion of the balloon sheath 220 extending distal to the proximal end of the microcatheter tube 210). In other words, when the detachable tip assembly 200 is coupled to the detachable balloon microcatheter 120, the balloon sheath 220 is sized so that it surrounds roughly half of the balloon 130.

In one embodiment, the balloon sheath 220 is constructed from a tube of highly-compliant elastomer such as polyurethane, silicone or PEBAX. However, other materials can be used.

The detachable tip sheathed balloon microcatheter 110 includes a detachment mechanism to allow the detachable tip assembly 200 to be selectively and controllably detached from the detachable balloon microcatheter 120 for the reasons discussed herein. The detachment mechanism can thus be operated by a surgeon to retract at least a portion (outer catheter tube 122) of the detachable balloon microcatheter 120 from the surgical site, while keeping the inner catheter tube and detachable tip assembly 200 fixed causing the atraumatic relates of the tip (with minimal strain on the vessels).

It will be understood that the aforementioned type of detachment mechanism is merely exemplary and there are any number of other mechanisms that can be used, under certain circumstances, to controllably detach the detachable tip assembly 200 from the detachable balloon microcatheter 120. For example, other suitable detachment mechanism can be in the form of those detachment mechanisms that are used in other catheter systems that require distal detachment (e.g., detachable tip microcatheters, embolic coils, other detachable embolic devices used outside the neurovascular market), and include electrolytic, piezoelectric, chemical, and mechanical separation mechanisms. It will also be appreciated that the detachment mechanisms can be in the form of a mechanical based mechanism (e.g., push/pull mechanism) or can be an electro-mechanical mechanism or other types of mechanism that is configured to controllably detach the microcatheter 120 from the assembly 200.

In one embodiment, the force that is required to detach the microcatheter 120 from the assembly 200 is less than the force that is attaching the cape/detachable portion of the embolic agent. As mentioned, the attachment/detachment mechanism can consist of either a press-fitting of the detachable tip structure into the interface of the catheter or a radially tensioned mechanism that can be adjusted by the surgeon to relieve radial clamping tension on the detachable tip and facilitate removal without any additional tension applied to the catheter. Other detachment mechanisms are described in detail below.

In the illustrated detachment mechanism, the balloon 130 is controlled by the clinician (surgeon) injecting fluid through the injection port 301 on the y-adaptor 300. It will be readily appreciated that since the protective balloon sheath 220 surrounds the balloon 130, the inflation and deflation of the balloon 130 directly controls movement of and the state of the balloon sheath 220. Thus, as shown in FIGS. 1 and 2, when the balloon 130 is fully inflated, the protective balloon sheath 220 is likewise outwardly extended and in an extended state and conversely, when the balloon 130 is fully deflated, the sheath 220 is in a collapsed state.

The protective balloon sheath 220 thus also expands and contracts with the balloon 130. Inflation and deflation of the balloon 130 allows flow control preventing reflux of embolic agent on non-targeted vessels. The balloon sheath 220 prevents attachment of Onyx™ or other cohesive or adhesive embolic agent directly to the balloon or to the detachment interface since the balloon sheath 220 covers the distal end of the balloon 130 which is the area that the embolic agent would contact in the event that the balloon sheath 220 was not present. The sheath 220 thus protects the balloon 130 from contact with the embolic agent, thereby eliminating the chance that the embolic agent contacts and bonds to the balloon material. In other words, the procedure is performed and controlled such that there is no reflux proximal to the sheath 220 or a radiopaque mark can be utilized to prevent the embolic agent (e.g., adhesive glue) from fixing the sheath 220 to the balloon 130.

As described in more detail herein, the embolic agent is delivered to the treatment site but delivering the embolic agent through the proximal hub into the inner catheter tube 124 and then subsequently into the microcatheter tube 210 from which it is ultimately discharged from the distal end of the microcatheter tube 210.

After completion of embolic agent delivery, the balloon 130 is deflated and the balloon 130 and the outer catheter tube 122 are retracted over the inner catheter tube 124 via rotation of thumb nut 320, releasing the detachable tip assembly 200 from the outer catheter tube 122 in the manner described herein.

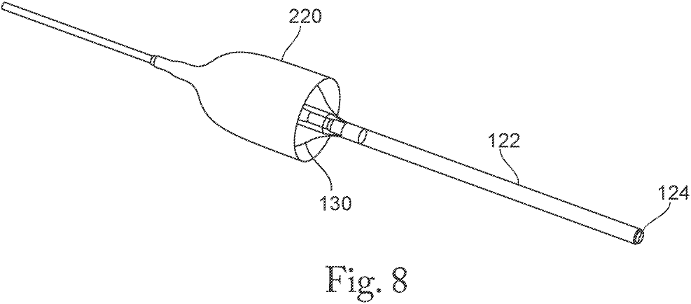

It will be understood that the detachable tip sheathed balloon microcatheter 110 can be constructed so as to have different embodiments than that shown in FIGS. 1-7. For example, and as illustrated in FIG. 8, the protective sheath 220 can be of an extended nature in that the tubular structure of the protective sheath 220 can be extended closer to the proximal end of the balloon 130, to help counteract the forward force on the tip that may otherwise be forced forward by the balloon 130 and detached prematurely. For example, the sheath 220 can cover 75% or more of the surface area of the balloon 130, or more than 80% or more than 90%.

In addition, one or more expanding wire structures (rings) can be embedded in a section of the protective balloon sheath 220 for helping to reduce this pushing force and provide extra friction between the balloon 130 and the balloon sheath 220 when the balloon 130 is expanded, further helping to prevent premature release of the detachable tip assembly 200.

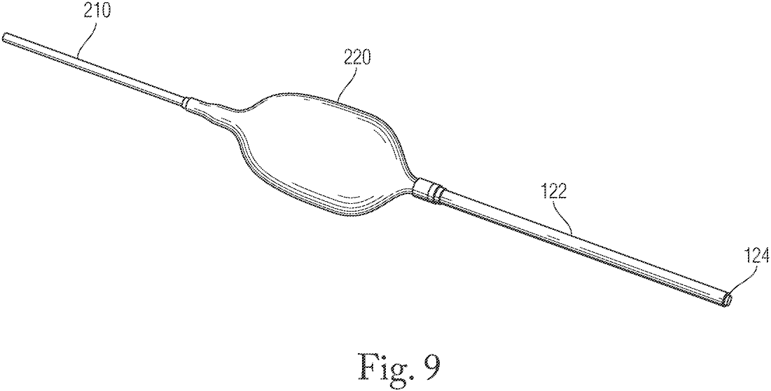

In another embodiment that is shown in FIG. 9, the elastomeric balloon sheath 220 is configured to surround the entire balloon (not shown), with the proximal end of the sheath 220 including a less-elastic and/or thicker ring section that resists expansion, preventing the balloon 130 from pushing the protective sheath 220 forward and detaching the tip assembly 200 prematurely.

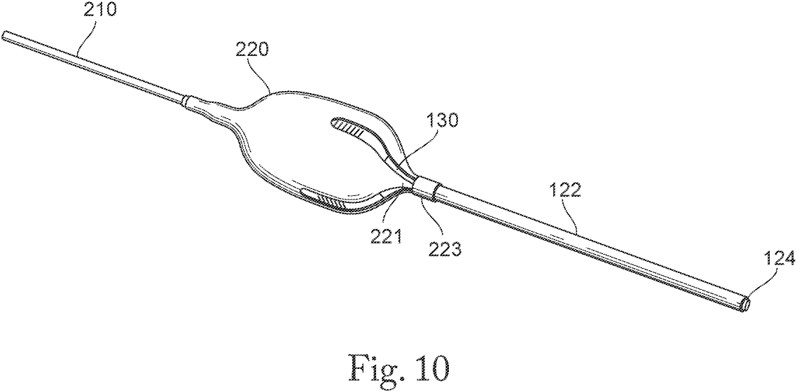

In yet another embodiment illustrated in FIG. 10, an elastomeric balloon sheath 220 covers the entire balloon 130, with the proximal end of the balloon sheath 220 being divided into strips 221 that are impinged by a ring 223 above the balloon seal. The ring 223 is pulled off of the strips 221 when the balloon microcatheter is retracted off of the tip, allowing the balloon microcatheter to slide out of the sheath 220.

In each of the exemplary embodiments described herein, the sheath 220 and the distal tip structure itself can be formed from polymeric materials with high affinity to form covalent bonds with ethylene vinyl alcohol copolymer, which is formulation of the Onyx™ adhesive embolic agent. This attraction between the sheath 220 and the Onyx™ adhesive embolic agent or other agent promotes the two to adhere to one another within the blood vessel such that detachment of the of the distal tip is achieved more cleanly. In addition, the material of the balloon 130 is preferably chemically dissimilar to the material that is used form the sheath 220 and the distal tip structure, thereby reducing the bonding affinity between the balloon 130 and the sheath 220/distal tip structure versus the Onyx™ adhesive embolic agent. In one embodiment, the sheath 220 is formed of ethylene and/or hydrophobic materials that adhere to the Onyx™ adhesive embolic agent and in one embodiment, the balloon 130 is formed of low-density polyethylene.

### Exemplary Method of Use

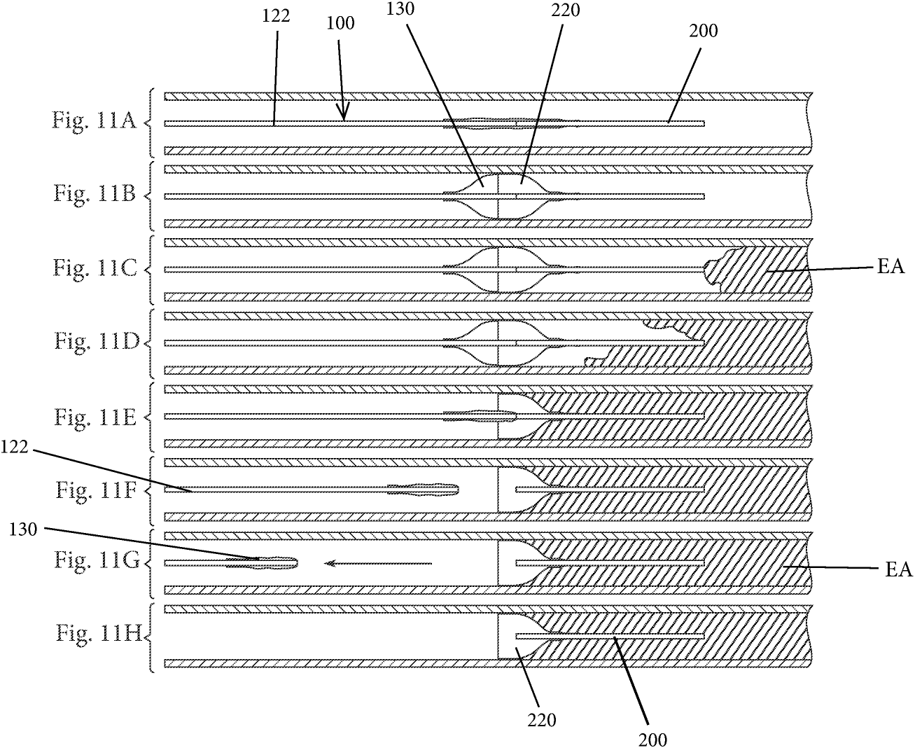

FIGS. 11A-11H show one exemplary use case of the detachable-tip balloon microcatheter system (assembly) 100 in the brain (e.g., in a vessel) and further illustrates its benefits compared to conventional balloon catheters and detachable tip catheters alone.

As shown in FIG. 11A, once the clinician identifies the AVM in the brain, the sheath-protected balloon microcatheter will be navigated within the target blood vessel. As shown in FIG. 11B, after microcatheter selection of target vessel, the clinician expands the balloon 130 to secure the device in the artery or vein either feeding or draining the nidus of the AVM, respectively. Expansion of the balloon 130 (by contrast material delivery thereto) causes synchronous expansion of the protective sheath 220. As shown in FIG. 11C, the clinician then begins to inject the embolic agent (e.g., Onyx ™)(identified as “EA”) by delivering the embolic agent through the inner catheter tube 124 (FIG. 1). As shown in FIG. 11D, reflux of the embolic agent from the injection of the embolic agent creates a cast that around the detachable tip assembly 200 effectively preventing reflux on normal vasculature. This step obviates the need for creation of a proximal plug by reflux alone of the embolic agent (e.g., Onyx ™) reducing the risk of complications described earlier (i.e., catheter entrapment) due to proximal reflux of the embolic agent (e.g., Onyx ™).

As shown in FIG. 11E, the clinician conducts a continuous AVM injection until the AVM is obliterated. Once the embolization procedure is complete (FIG. 11F), the clinician deflates the balloon 130 and as shown in FIG. 11G, the clinician mechanically retracts the outer catheter tube 122 with balloon 130 over the inner catheter tube 124 separating the detachable tip assembly 200 from the detachable balloon microcatheter 120. As shown in FIG. 11H, the detached tip assembly 200 remains firmly embedded in the embolic agent (Onyx ™) cast. Finally, the clinician removes the main catheter from the patient.

As shown in FIGS. 11A-11H, the protective balloon sheath 220 prevents dangerous reflux of the embolization agent and spillage into adjacent blood vessels. The balloon sheath 220 facilitates the formation of a cast around the microcatheter tip (assembly 200). The entire procedure facilitates comprehensive injection of the embolic agent prevents catheter entrapment and reduces procedure time.

It will be understood that process depicted in FIGS. 11A-11H does not specify if access to the AVM is made through the artery or through a vein. The present invention described herein can be utilized in both transvenous and transarterial embolization procedures.

For sake of simplicity, the inner catheter tube 124 is not shown in FIGS. 11A-11H.

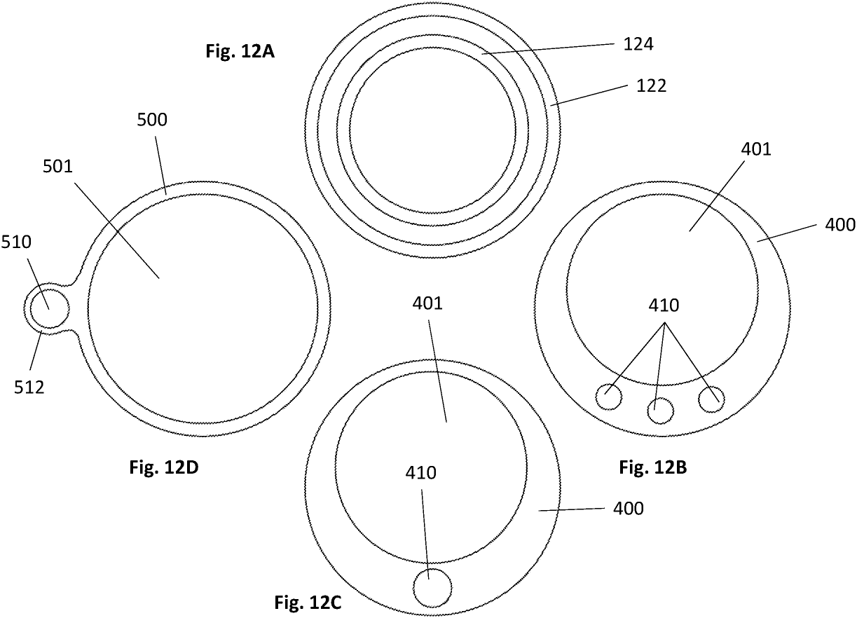

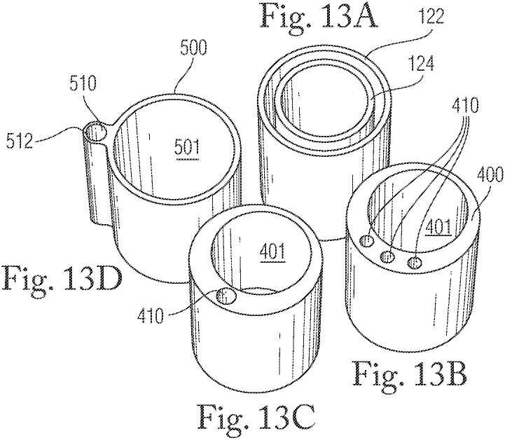

FIGS. 12A-12D are cross-sectional views of alternative detachable tip sheathed balloon microcatheters that can be used in accordance with the practice of the present invention. FIGS. 13A-13D are perspective views of the microcatheters shown in FIGS. 12A-12D. In general, the microcatheter includes two lumens, namely, a first lumen for the embolic agent and one for the inflation fluid for controlling the inflation and deflation of the balloon. FIGS. 12A and 13A illustrate the previously described embodiment in which the catheter comprises the outer catheter tube 122 and the inner catheter tube 124, with the inflation fluid (e.g., saline) passing between the inner diameter of the outer catheter tube 122 and the outer diameter of the inner catheter tube 124. FIGS. 12B and 13B illustrate a microcatheter that is formed of a catheter body 400 that includes a main lumen 401 that is preferably centrally located and a plurality of secondary lumens 410 that are formed within the catheter body 410. While the secondary lumens 410 are shown as having the same shape and size (circular with same diameter), it will be appreciated that lumens 410 can have difference shapes and/or sizes. Also, the lumens 410 are shown as generally being grouped together; however, the lumens 410 do not have to grouped together and one or more of the lumens can be separated from the others. The secondary lumens 410 are located adjacent the main lumen 401. FIGS. 12C and 13C show an embodiment in which there is only a single secondary lumen 410 formed in the catheter body 410. FIGS. 12D and 13D illustrate a microcatheter formed of a catheter body 500 with a main lumen 501 formed therein. In this embodiment, a secondary lumen 510 is defined within a tubular structure 512 that is located adjacent the main lumen 501. As shown, the tubular structure 512 extends longitudinally along the main catheter body 500 and can be integrally formed therewith, as illustrated, or can be a separate part that is coupled to the main catheter body 500 using traditional techniques. The secondary lumen 510 is intended to carry the balloon inflation fluid.

It will also be appreciated that other microcatheter constructions can be used in accordance with the present invention and the ones disclosed and described herein are only exemplary and not limiting of the present invention.

It will be appreciated that the inclusion of the sheath 220 in the current system serves several purpose in that the sheath 220 shields the catheter balloon but it does more than that in that without the sheath 220, detachment of the two subassemblies would not be generally possible since the embolic agent (e.g., Onyx™) would reflux onto the detachment mechanism and thereby bind such system and prevents detachment of these two subassemblies. In other words, the sheath 220 not only provides shielding (protection) of the balloon 130 but it also shields (protects) the detachment mechanism and prevents the embolic agent from contacting the detachment mechanism and binding to it so as to render it inoperable. The incorporation of sheath 220 enables a short throw of the distal end effector of the device, further compacting its function so the entire device is as proximal as possible to the disease (target site), which improves efficacy. The protected balloon 130 is therefore enabled to do what balloons do best, namely, control and occlude flow dynamically at the target site.

The present invention is directed to systems and methods for treatment of patients diagnosed with arteriovenous malformations. The improved technology described herein increases the efficiency of embolization, reduces undesirable complications due to reflux, insufficient embolization of nidus, and entrapment of the catheter. One exemplary embodiment of the new technology is a single body double lumen microcatheter that is DMSO and NBCA compatible, includes a detachable-tip and balloon protective sheath and makes unnecessary the double catheter techniques applied to conventional transvenous and transarterial catheterization. As mentioned herein, the present technology can be extended to embolization of non-cerebral AVMs. The present improved technology will improve the health outcomes of patients with AVMs by facilitating complete occlusion and limiting procedural risks. Furthermore, condensing the dual-catheter embolization technique into a single device will allow for improved time efficiency intra-procedurally and overall improvement in healthcare cost savings.

It will also be appreciated that while a balloon microcatheter is described herein as being one exemplary means for expanding the sheath, other means can be used to cause an expansion (opening) of the sheath. For example, a catheter with a mechanical device or mechanism at the distal end can be used and upon actuation, the device is configured to apply an outward radial force to the sheath to cause the sheath to open in the manner described herein. In addition, the use of memory materials can be used to radially expand the sheath. For example, in an at rest position, the memory material can have an expanded annular shape for contacting and driving the sheath to the open position; however, the memory material can be causes to collapse into a collapsed state as by a mechanical mechanism (actuator). Other devices/actuators can be used to cause the controlled radial expansion of the sheath, while still allowing delivery of the embolic agent to the target location as by passage through a center lumen. In these other embodiments, the sheath also shields these devices from the embolic agent that is ejected to allow for detachment of the detachable tip part that contains the sheath from the other device.

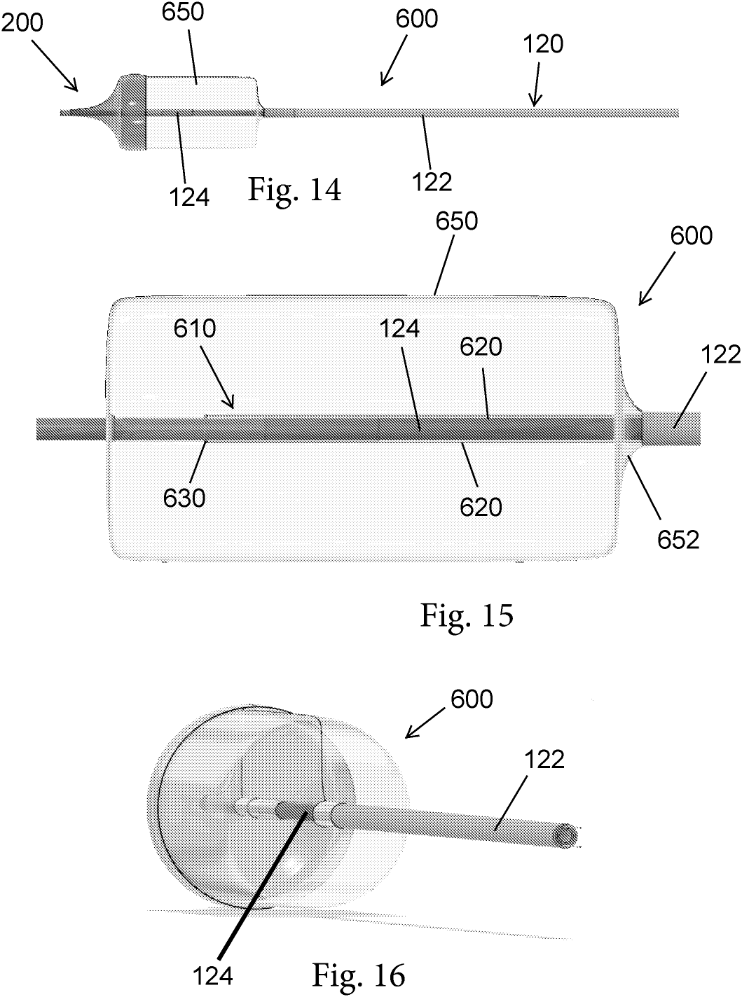

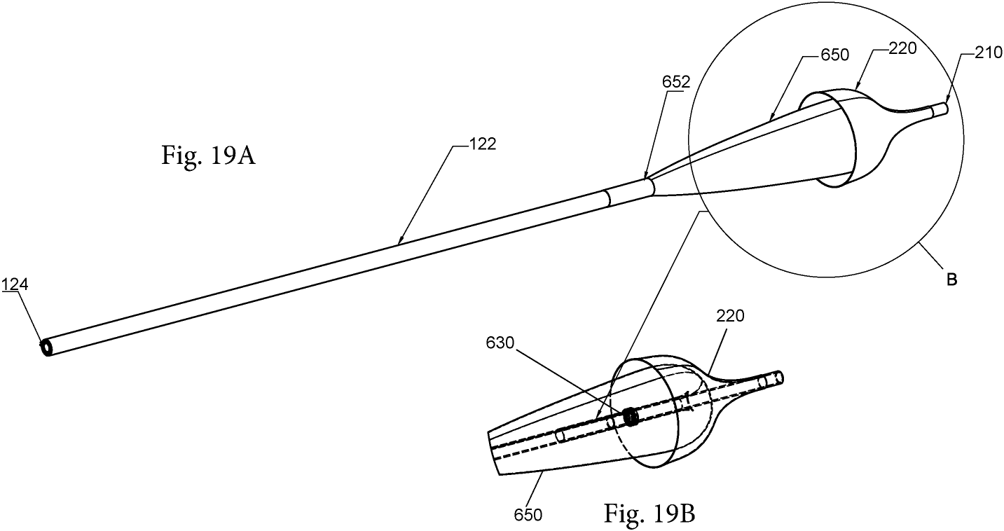

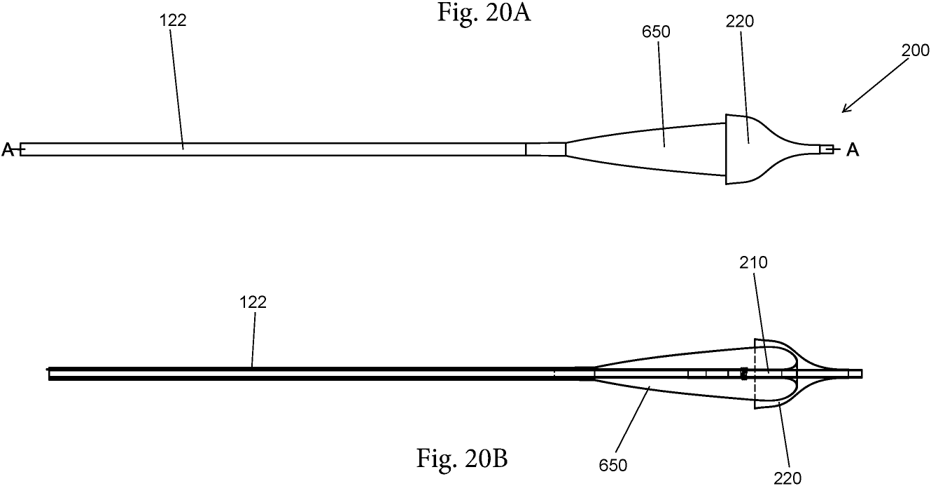

### Detachment Mechanism (FIGS. 14-22)

As discussed herein, a detachment mechanism is provided as part of the overall system in order to detachably couple the detachable tip portion to the main balloon microcatheter. It will be understood that the detachment mechanism described herein and illustrated in the figures (e.g., FIGS. 14-22) is not limited to being used with a detachable tip assembly 200 that includes the sheath 220 but instead can be used with many different types of detachable tips that are detachably coupled to a distal end of the main microcatheter or other main catheter device or the like. Thus, the sheathed detachable tip described and illustrated herein is merely exemplary and not limiting of the scope of the present disclosure and in particular, is not limiting of the use and application of the detachment mechanism described herein. In addition, while the main microcatheter (e.g., microcatheter 120) is described as being a balloon microcatheter, it will be appreciated that the detachment mechanism described herein can likewise be used with other types of microcatheters includes those that do not include a balloon, etc.

In general, the present disclosure an improved detachment mechanism that can be used to selectively couple a distal detachable tip component to a main body, which can be a microcatheter. Once at a target site, the detachment mechanism can be actuated resulting in the distal detachable tip component being detached from the main body.

FIGS. 14-22 illustrate one exemplary detachable tip balloon microcatheter system 600 that is similar to the detachable-tip balloon microcatheter system (assembly) 100 and therefore like elements are numbered alike. Like the system 100, the system 600 can comprise a detachable tip sheathed balloon microcatheter that is formed of two main parts, namely, the detachable balloon microcatheter 120 (first part of both the systems 100 and 600) and the detachable tip assembly 200 (second part of the system 600) with a detachment mechanism 610 serving to detachably couple the two parts together. The detachment mechanism 610 is thus constructed so that in a first operating state, the detachment mechanism 610 maintains the first part of the system 600 (e.g., the detachable balloon microcatheter 120) in an attached state to the second part of the system 600 (e.g., the detachable tip assembly 200) and in a second operating state, the detachment mechanism 610 allows for separation (detachment) of the two parts 120, 200, thereby allowing the detachable tip assembly 200 to be left behind while at least a portion of the detachable balloon microcatheter 120 is removed from the site.

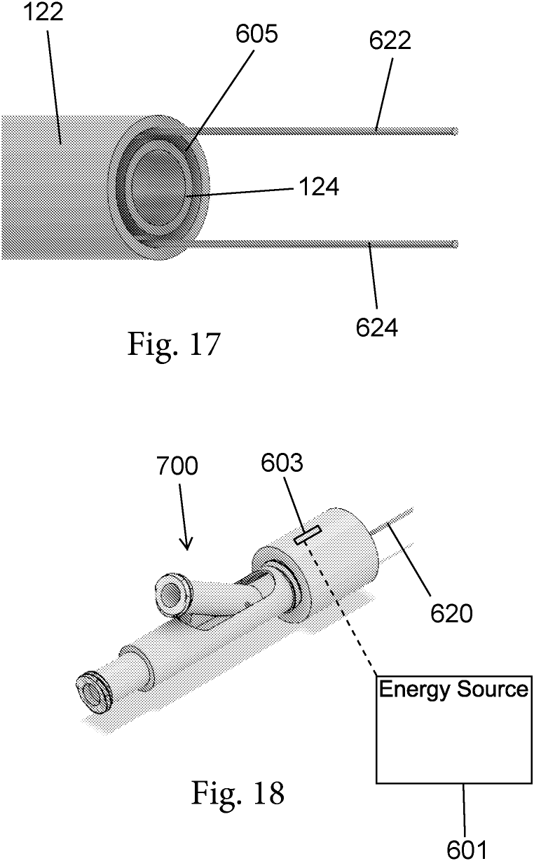

As shown in FIG. 17, a space 605 is formed between the inner catheter tube 124 and the outer catheter tube 122. This space 605 can have an annular shape when the tubes 122, 124 are cylindrical in shape. This space 605 provides for a longitudinal passageway in which at least part of the detachment mechanism 610 can be located. In the illustrated embodiment, the detachment mechanism 610 comprises a shaped memory wire 620 that is routed through the space 605 as discussed herein and has a coiled portion 630 at a distal end that this configured to releasably hold and retain the detachable tip assembly 200. The wire 620 is a shape memory wire that is formed of a shape memory material, such as a shape memory alloy that is an alloy that can be deformed when cold but returns to its pre-deformed (“remembered”) shape when heated. In one embodiment, the wire 620 is formed of nitinol which is an alloy with a composition of equal parts of nickel and titanium. It will be appreciated that other types of wires 620 can be used.

In one embodiment, the wire 620 has a diameter of about 0.005 inch (0.127 mm); however, this value is merely exemplary and not limiting of the scope of the present disclosure since the wire 620 can equally be formed to have other diameters. The diameter of the wire 620 is selected in view of the size of the space 605. The nitinol wire’s purpose, in shape memory, is to expand under heat (e.g., 30 - 130° C.) or current (e.g., 200 mA). As a super elastic wire, the wire 620 can be stretched (decrease in diameter) and under the same application of current or heat, will induce the recovery of its diameter.

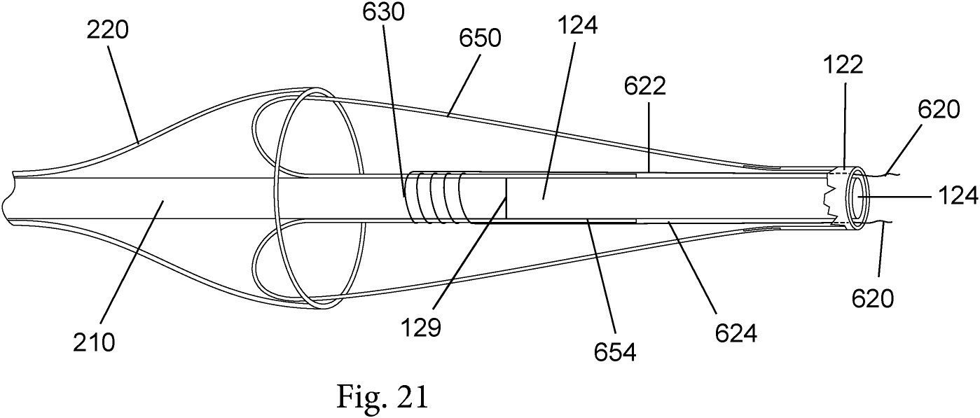

The wire 620 can have a first longitudinal section 622 that extends longitudinally within the space 605 between the two tubes 122, 124 and a second longitudinal section 624 that likewise extends longitudinally within the space 605 between the two tubes 122, 124 (See, FIGS. 17 and 21). The first longitudinal section 622 is circumferentially spaced from the second longitudinal section 624. One of the longitudinal sections 622, 624 can be considered to represent the wire 620 traveling toward the coiled portion 630, while the other longitudinal section 622, 624 can be considered to represent the wire 620 traveling away from the coiled portion 630. In one embodiment, the first longitudinal section 622 and the second longitudinal section 624 are located about 180 degrees apart and thus, run parallel to one another at least along some lengths of the wire sections.

Since the space 605 is located between the inner catheter tube 124 and the outer catheter tube 122, the first and second longitudinal sections 622, 624 travel within the catheter structure itself and exit at the distal end of the outer catheter tube 122. As shown in the figures, the inner catheter tube 124 extends distal to the outer catheter tube 122 and therefore has an exposed length to which one end (inverted end) of the balloon is attached, as described below, and also over which the wire 620 is routed.

As mentioned, the first and second longitudinal sections 622, 624 terminate at the coiled portion 630 at which the wire is wound in a number of turns (windings) to define the coiled portion 630. The number of windings can vary depending upon the application and the number of windings can, for example, be between 2 and 6. However, other number of windings are equally possible.

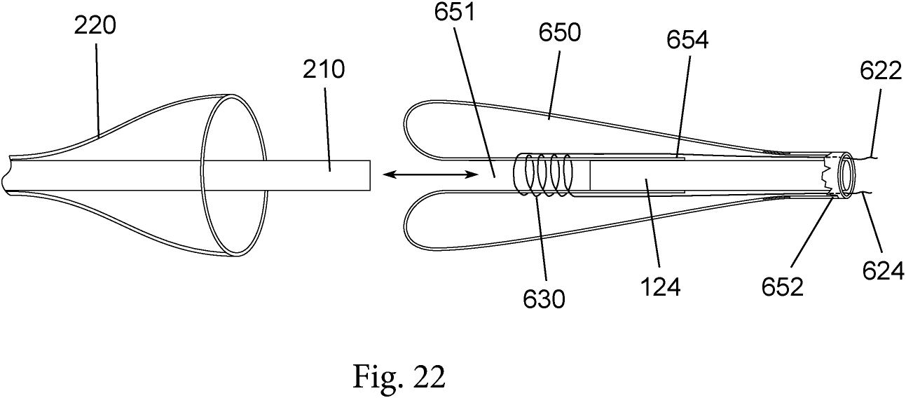

As shown in FIGS. 14-22, the system 600 has a balloon 650 (which can be the same or similar to balloon 130) that is fixedly coupled to the outer catheter tube 122 (e.g., at a distal end thereof) and to an exposed distal end portion of the inner catheter tube 124 which represents a length of the inner catheter tube 124 that is distal to and is exposed beyond the distal end of the outer catheter tube 122. As best shown in FIGS. 21 and 22, the balloon 650 thus has a first end 652 and a second end 654. The first end 652 of the balloon 650 is attached to the outer surface of the outer catheter tube 122 a first location such as the distal end of the outer catheter tube 122. The balloon 650 is inverted and folded into itself as shown such that the second end 654 is attached to the outer surface of the inner catheter tube 124 at a second location that is distal to the first location and distal to the distal end of the outer catheter tube 122 but is proximal to the distal end (identified at 129 in FIG. 21) of the inner catheter tube 124. By forming an inverted distal end portion of the balloon 650, an open space (hollow center space) 651 is formed. In other words, this inverted distal end portion of the balloon 650 can have an annular shape with a center opening 651. The depth of this center opening 651 can vary depending upon the dimensions of the balloon 650 and the other components. The center opening 651 thus terminates at the distal end of the inner catheter tube 124.

It will also be appreciated that the space 605 between the inner catheter tube 124 and the outer catheter tube 122 represents an inflation lumen through which inflation medium flows in order to inflate and deflate the balloon 650. Since the first end 652 of the balloon 650 is attached to the outer catheter tube 122 and the second end 654 of the balloon 650 is attached to the inner catheter tube 124, the wire 620 that exits the space 605 will be located inside of the balloon 650 within its inverted annular shaped portion.

The placement of the wire 620 is very important for its ideal operation. The placement of the balloon 650 is optimized for this detachment mechanism where the balloon 650 ending on the distal end (detachment part 200) is inverted and overlapping the outer catheter tube 122, shown in FIGS. 21 and 22. The wire 620 is wrapped at a diameter slightly larger to the diameter of the (inverted portion) of the balloon 650 over the outer catheter tube 122. The wire 620 is then preheated to its annealing phase at 500° C. to remember the shape. Once annealing is complete, the wire 620 is tightly wrapped over the inverted portion of the balloon 650 on the detachable tip end, not allowing the detachment until the wire 620 is activated to expand as discussed herein.

In the illustrated detachable tip assembly 200, the microcatheter tube 210 is sized and configured to be received within the open space 651 that is formed within the inverted portion of the balloon 650 and can be brought into an abutting relationship with the distal end of the inner catheter tube 122 as shown in FIG. 21 (i.e., the two abut at 129). The microcatheter tube 210 can thus pass through the coiled portion 630 with the balloon material being disposed between the microcatheter tube 210 and the coils of the coiled portion 630 since wire 620 including the coiled portion 630 are located inside the balloon 650. It will be understood that in embodiment in which the microcatheter does not include the balloon 650, the coiled portion 630 can be placed into direct contact with the detachable tip assembly 200. In other words, in the closed position, the coiled portion 630 is wound tightly around and directly in contact with the detachable tip assembly 200 (e.g., the catheter 210 thereof).

In the first operating state of the detachment mechanism 610 shown in FIG. 21, the wire 620 and coiled portion 630 thereof are in a closed state in that the coiled portion 630 is tightly wound around the microcatheter tube 210 (with the balloon material disposed therebetween), thereby holding the microcatheter tube 210 in place relative to the detachable balloon microcatheter 120. The coiled portion 630 thus applies a grasping force to the tube 210 that effectively grasps and holds the tube 210 within the tight coiled portion 630.

In the second operating state, the coiled portion 630 is expanded radially outward so as to open and release the microcatheter tube 210 from its grasp and thereby allowing the detachable tip assembly 200 to be fully separated from the detachable balloon microcatheter 120. As described herein, the detachment mechanism 610 thus allows the detachable tip assembly 200 (parts 220, 210) to be left behind at the site while the detachable balloon microcatheter 120 is removed.

The detachment mechanism 610 can be activated, by a physician, to expand inside the body post-embolization. As mentioned, the wire 620 feeds from the saline lumen (space 605), through the catheter tubing, to the outside of a rotating hemostatic valve (RHV) 700 (FIG. 18) and can be attached to an energy reservoir (energy source 601) designed as simple, but not limited to a battery with a switch 603 as shown in FIGS. 16-18. In other words, the RHV 700 can include a controller or actuator 603 which is designed to control operation of the applied current to the wire 620 and can be in the form of a switch, button, etc. Controlled timing of optimal wire activation (to avoid complications during surgery) can be calculated and controlled (programmed). Once the determined optimal timing is completed, the switch can be turned off and/or battery removed to stop heating of the wire 620. The cooling of the wire 620 causes it to transform back to its remembered state to allow for subsequent retraction of the detachable balloon microcatheter 120. In the figure, the energy source 601 is shown separated from the actuator 603, however, it will be appreciated that the energy source 601 can be a battery that can be incorporated into and disposed within the handle body itself (e.g., RHV body 700). Alternatively, the handle can include a port to which a cable can be attached for electrically connecting the energy source 601 to the actuator 603 for providing electrical current to the wire 620.

### Exemplary Operation of the Detachment Mechanism

Once the detachable tip balloon microcatheter system 600 is guided and placed in a desired (target) location in the vasculature and embolization has already taken place, the next step is to detach the catheter tip with sheath (to be an implant) (e.g., detachable tip assembly 200) and retract the remainder of the catheter (microcatheter 120) from the body. The following are exemplary steps of operation:

1. While balloon 650 is still inflated and the embolic material (embolic agent) has hardened, turn on the switch 603 or press the button 603 at the end of the RHV 700 to activate the current passing through the wire 620, allowing expansion, (reminder - make sure the energy source 601 (e.g., battery) is in the proper place).

2. Allow the appropriate timing (to be determined based on various operating parameters) for guaranteed expansion of the wire 620, then turn off switch 603 or press the button 603 again to stop current flow.

3. As a potential safety factor, detachment of the power source 601 can occur to prevent continuous current which could damage the device (system 600) and potentially the patient.

4. Retract the catheter (microcatheter 120) slowly. If resistance is experienced, repeat steps 1 and 2, then retract catheter (microcatheter 120). The detachable tip assembly 200 is left behind.

5. Dispose of catheter with wire (microcatheter 120). Keep the reusable power source 601 for future procedures.

In certain embodiments in which the first and second longitudinal sections 622, 624 of the wire 620 run along the outer surface of the inner catheter tube 124 and the inverted portion of the balloon 650 that is seated against the inner catheter tube 124, a covering material can be disposed over at least a portion of the first and second longitudinal sections 622, 624 to hold these sections in place without disrupting the heating thereof. For example, a suitable tape segment or similar structure can be placed over one or more locations along the first and second longitudinal sections 622, 624 for securing the wire 620 in place.

The unique shape memory characteristics of the wire 620 provides a means for selectively: (1) grasping and holding the detachable tip assembly 200 and more specifically, the microcatheter tube 210 thereof; and (2) releasing the detachable tip assembly 200 from the detachable balloon microcatheter 120 by expanding. The disclosed detachment mechanism 610 is easy to operate and simple in construction and can easily be implemented in existing products.

It is to be understood that like numerals in the drawings represent like elements through the several figures, and that not all components and/or steps described and illustrated with reference to the figures are required for all embodiments or arrangements. Further, the terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components, and/or groups thereof.

It should be noted that use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Overall, the subject matter described above is provided by way of illustration only and should not be construed as limiting. Various modifications and changes can be made to the subject matter described herein without following the example embodiments and applications illustrated and described, and without departing from the true spirit and scope of the present invention, which is set forth in the following claims.

What is claimed is:

1. An endovascular device comprising:
a catheter;
a detachment mechanism comprising a shape memory material located at a distal end of the catheter; and
a detachable tip component that is detachably secured to the catheter by the detachment mechanism, wherein the detachment mechanism is configured such that application of electricity to the detachment mechanism causes the detachment mechanism to open and release the detachable tip component.

2. The device of claim 1, wherein the shape memory material is nitinol.

3. The device of claim 1, wherein the detachment mechanism comprises a coil that is formed of the shape memory material.

4. The device of claim 3, wherein the coil surrounds the detachable tip component and is distally spaced from the catheter.

5. The device of claim 4, wherein in a rest position of the detachment mechanism, when the shape memory material, including the coil, is not under influence of electricity, the coil tightly grasps the detachable tip component, which passes through an opening of the coil, and maintains coupling between the embolic device and the catheter and wherein in an actuated state when electricity is applied, the coil expands and opens, thereby releasing the detachable tip component and severing the coupling between the detachable tip component and the catheter.

6. The device of claim 1, wherein the catheter comprises a balloon catheter including an outer catheter and an inner catheter that is disposed internally within the outer catheter, the inner catheter extending distal to the outer catheter and wherein a balloon is coupled to the outer catheter.

7. The device of claim 6, wherein the shape memory material is disposed between the outer catheter and the inner catheter in a lumen in which balloon inflation fluid is disposed.

8. The device of claim 6, wherein the balloon is configured such that a distal end of the balloon that surrounds the detachable tip component is inverted and overlaps the catheter.

9. The device of claim 1, wherein in an at rest position when electricity is not applied to the shape memory material, the detachable tip component abuts the catheter and conversely in an actuated state, when electricity is applied to the shape memory material, the detachable tip component is free to separate from the catheter.

10. The device of claim 9, wherein the detachable tip component includes a shaft that abuts the catheter, wherein a diameter of the shaft and a diameter of the catheter are approximately equal.

11. The device of claim 1, wherein the shape memory material comprises a coil that is tightly wound about a shaft that is part of the detachable tip component.

12. The device of claim 1, wherein the detachable tip component comprises an embolic device.

13. The device of claim 1, wherein the shape memory material of the detachment device has: (1) a longitudinal portion that extends along a length of the catheter and a length of the embolic device and (2) a coil portion that is only disposed about the detachable tip component.

14. The device of claim 13, wherein the longitudinal portion comprises a first longitudinal segment and a second longitudinal segment that is circumferentially spaced relative to the first longitudinal segment.

15. The device of claim 13, wherein the coil portion has at least one winding disposed about the detachable tip component in an at rest position prior to application of electricity.

16. The device of claim 13, wherein the coil portion has two or more windings disposed about the detachable tip component in an at rest position prior to application of electricity.

17. The device of claim 1, wherein the catheter includes a handle that contains an actuator for applying the electricity to the detachment mechanism.

18. The device of claim 1, further include a battery which provides electric current that flows through the shape memory material and causes expansion thereof and opening of a coil portion of the detachment mechanism that in a rest state is tightly wound about the detachable tip component.

19. The device of claim 1, wherein the catheter comprises a balloon catheter with the detachable tip component being at least partially received within the balloon.

20. The device of claim 1, wherein a proximal portion of the detachment mechanism is disposed within an annular shaped lumen formed in the catheter and a distal end portion of the detachment mechanism comprises a coiled portion that is disposed about the detachable tip component and not the catheter.

21. The device of claim 1, wherein the embolic devices comprises a distal tip member with a shaft that abuts a distal end of the catheter in an at rest position when electricity is not applied to the shape memory material, and conversely in an actuated state, when electricity is applied to the shape memory material, the distal tip member is free to separate from the distal end of the catheter.

22. An endovascular device comprising:
a catheter having a distal end;
a detachable tip component that is disposed distal to the distal end of the catheter;
a detachment mechanism for detachably coupling the detachable tip component to the catheter, the detachment mechanism being in contact with the detachable tip component at an attachment point and comprising a wire formed of a shape memory material, the detachment mechanism being configured such that application of electrical current or heat to the wire causes a coil, that is formed at a distal end of the wire, to expand and open, thereby releasing the detachable tip component from the detachment mechanism and allowing separation of the detachable tip component from the catheter.

23. The device of claim 22, wherein the catheter includes an outer catheter tube and an inner catheter tube that is disposed inside a lumen of the outer catheter tube and extends distally beyond the outer catheter tube, the catheter further including a balloon that has a first end attached to the outer catheter tube and a second end attached to the inner catheter tube and an inverted portion that is located distal to the inner catheter tube, the inverted portion having a center opening for receiving the detachable tip component.

24. The device of claim 23, wherein the coil of the wire is disposed around the inverted portion of the balloon, the balloon being disposed between the coil and a proximal section of the detachable tip component, wherein in an at rest position, the coil is tightly wound about the proximal section and in an open position, the expanded coil permits the proximal section to be released.

25. A method for detaching a detachable tip component that is selectively coupled to a catheter of an endovascular device comprising the steps of:
applying electric current or heat to a detachment mechanism that selectively couples the detachable tip component to the catheter causing the detachment mechanism to open and release the detachable tip component allowing separation of the detachable tip component from the catheter, wherein the detachment mechanism includes a shape memory material.

26. The method of claim 25, wherein the detachment mechanism comprises a coil that is formed of the shape memory material and the step of applying electric current or heat comprises the step of applying electric current or current to the coil to cause expansion of the coil, thereby allowing separation and detachment of the detachable tip component from the catheter.

27. The method of claim 26, wherein the coil surrounds and wraps around the detachable tip component and is distally spaced from the catheter.

28. The method of claim 27, wherein the coil is a plurality of windings wound about a shaft of the detachable tip component.

29. The method of claim 25, wherein the shape memory material of the detachment device has: (1) a longitudinal portion that extends along a length of the catheter and a length of the detachable tip component and (2) a coil portion that is only disposed about the detachable tip component.

30. The method of claim 29, wherein the longitudinal portion comprises a first longitudinal segment and a second longitudinal segment that is circumferentially spaced relative to the first longitudinal segment.

31. An endovascular device comprising:
a main body;
a detachment mechanism comprising a shape memory material located at a distal end of the main body; and
a detachable tip component that is detachably secured to the main body by the detachment mechanism, wherein the detachment mechanism is configured such that application of electricity to the detachment mechanism causes the detachment mechanism to open and release the detachable tip component from the main body.
